(19) United States
(12) Patent Application Publication
(10) Pub. No.: US 2026/0041445 A1
(43) Pub. Date: Feb. 12, 2026

(54) LITHOTRIPSY BALLOON CATHETER

(71) Applicant: Medtronic, Inc., Minneapolis, MN (US)

(72) Inventors: Bradley E. Steele, Plymouth, MN (US); Thomas J. McPeak, Caledonia, MN (US); Diane M. Haen, Blaine, MN (US)

(21) Appl. No.: 19/105,459

(22) PCT Filed: Aug. 31, 2023
(86) PCT No.: PCT/US2023/031600

§ 371 Date: Feb. 21, 2025

Related U.S. Application Data
(60) Provisional application No. 63/374,014, filed on Aug. 31, 2022.

(51) Int. Cl. A61B 17/22; A61B 17/00

(52) U.S. Cl. CPC A61B 17/22022; A61B 17/00234; A61B 2017/00238; A61B 2017/00305; A61B 2017/00367; A61B 2017/00557; A61B 2017/00929; A61B 2017/22038

(57) ABSTRACT

A lithotripsy balloon catheter may include a shock wave emitter that is selectively movable longitudinally within the balloon to adjust a longitudinal position of the shock wave emitter relative to the balloon. A lithotripsy balloon catheter may include a unipolar electrode that produces an electrical arc when a voltage is applied to the unipolar electrode thereby creating a shock wave within the balloon. A grounding conductor for the shock wave emitter may be coupled to the proximal end portion of the catheter body and configured to be connected to ground. A lithotripsy balloon catheter may include a unipolar electrode in communication with an electrical source of energy and configured to deliver energy from the electrical energy source to the fluid in the balloon thereby creating a shock wave within the balloon. Ceramic insulation may be disposed on the unipolar electrode to focus energy at a tip of the unipolar electrode.

### FIELD

The present technology is generally related to a lithotripsy balloon catheter and system including the same.

### BACKGROUND

An intravascular lithotripsy (IVL) balloon catheter may be used to break up a calcified lesion within a patient's vasculature. The IVL balloon catheter may have a balloon, such as an angioplasty balloon, at the distal end thereof arranged to be inflated with a fluid. A shock wave emitter is received in the balloon. When the inflated balloon is placed adjacent a calcified region of a vein or artery a shock wave is formed in the balloon that propagates through the fluid and impinges upon the wall of the balloon and the calcified lesion. Repeated pulses break up the calcium.

### SUMMARY

The techniques of this disclosure generally relate to a lithotripsy balloon catheter, such as an intravascular lithotripsy balloon catheter.

In one aspect, the present disclosure is directed to a lithotripsy balloon catheter system comprising a catheter body configured to be received in a lumen of a subject. The catheter body has opposite proximal and distal end portions and a longitudinal axis extending between the proximal and distal end portions. An energy source is coupled to the catheter body. A balloon is coupled to the distal end portion of the catheter body. The balloon is configured to receive fluid to expand the balloon. A shock wave emitter is in the balloon. The shock wave emitter is configured to receive energy from the source of energy and configured to use the received energy to create a shock wave that propagates through the fluid in the balloon. The shock wave emitter is selectively movable longitudinally within the balloon to adjust a longitudinal position of the shock wave emitter relative to the balloon.

In another aspect, the disclosure is directed to a lithotripsy balloon catheter comprising a catheter body configured to be received in a lumen of a subject. The catheter body has opposite proximal and distal end portions and a longitudinal axis extending between the proximal and distal end portions. An electrical energy source is coupled to the catheter body. A balloon is coupled to the distal end portion of the catheter body. The balloon is configured to receive fluid to expand the balloon. A shock wave emitter is in the balloon. The shock wave emitter includes a unipolar electrode in communication with the electrical source of energy and configured to deliver energy from the electrical energy source to the fluid in the balloon thereby creating a shock wave within the balloon. A ceramic insulation is disposed on the unipolar electrode to focus energy at a tip of the unipolar electrode.

In yet another aspect, the disclosure is directed to a lithotripsy balloon catheter comprising a catheter body configured to be received in a lumen of a subject. The catheter body has opposite proximal and distal end portions and a longitudinal axis extending between the proximal and distal end portions. A balloon is coupled to the distal end portion of the catheter body. The balloon is configured to receive fluid to expand the balloon. A shock wave emitter is received in the balloon. The shock wave emitter comprises a unipolar electrode. The unipolar electrode is configured to produce an electrical arc when a voltage is applied to the unipolar electrode thereby creating a shock wave within the balloon. A grounding conductor for the shock wave emitter coupled to the proximal end portion of the catheter body and configured to be connected to ground.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a schematic representation of a first embodiment of an intravascular lithotripsy (IVL) balloon catheter system.

FIG. 2 is an enlarged, cross section of a distal end portion of a balloon catheter of the intravascular lithotripsy (IVL) balloon catheter system.

FIG. 3 is similar to FIG. 2, showing the distal end portion of the balloon catheter inside a body lumen.

FIG. 4 is an enlarged, cross section of a distal end portion of a balloon catheter of a second embodiment of an intravascular lithotripsy (IVL) balloon catheter system.

FIG. 5 is similar to FIG. 4, showing the distal end portion of the balloon catheter inside a body lumen.

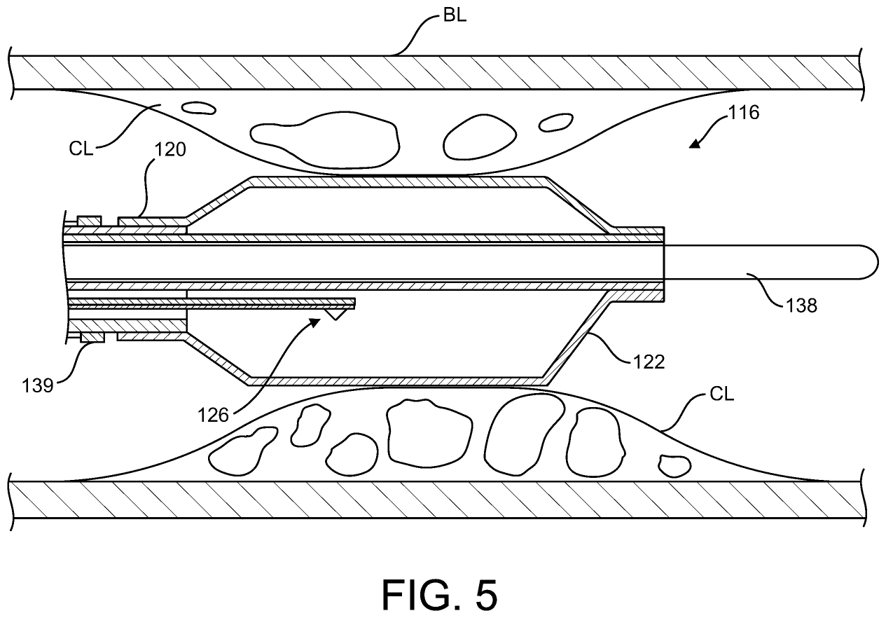

### DETAILED DESCRIPTION

In general, the present description is directed to a balloon catheter configured to modify calcified lesions through lithotripsy. The balloon catheter includes a shock wave emitter inside a fluid-filled balloon of the catheter. The shock wave emitter is configured to delivery energy to the fluid of the balloon. The delivered energy is suitable to produce a shock wave in the fluid that propagates through the balloon to the calcified lesion. In one example, the shock wave emitter may be configured to deliver electrical energy, electromagnetic energy (e.g., laser or light energy), mechanical energy, or other types of suitable energy to the fluid to generate a shock wave for treating the calcified lesion.

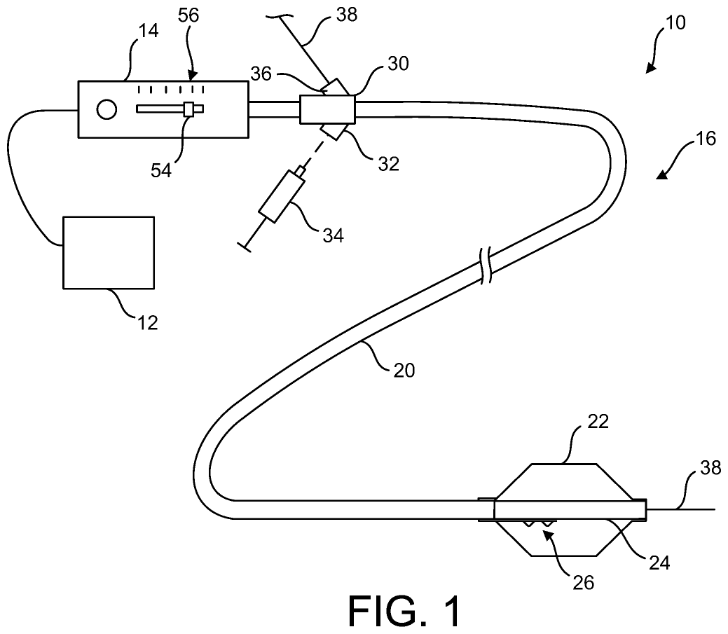

Referring to FIG. 1, one embodiment of an intravascular lithotripsy (IVL) balloon catheter system is generally indicated at reference numeral 10. In general, the IVL balloon catheter system 10 includes an energy source 12, a control handle 14 coupled to the energy source, and an IVL balloon catheter, generally indicated at 16, coupled to the control handle. The illustrated IVL balloon catheter system 10 is configured to modify calcified lesions within blood vessels of a patient. It is contemplated that the teachings of the present disclosure may be used to treat other body lumens and structures (e.g., heart valve) for breaking up lesions (e.g., calcified lesions) or other obstructions. The illustrated energy source 12 may be an electrical energy source (e.g., a voltage generator, such as a high voltage pulse source). In the below disclosure, the energy source is described as a high voltage (HV) source, with the understanding that the energy source is not limited to a HV source in one or more embodiments. The HV source may include a single voltage, direct current source, generating from about 100 volts to about 3000 volts, for example. In other embodiments, the energy source may be an electromagnetic source (e.g., a laser) or other type of energy sources.

Referring still to FIG. 1, the IVL balloon catheter 16 generally includes an elongate catheter body 20 having a longitudinal axis; an expandable balloon 22 (shown expanded in FIGS. 1-3) at the distal end portion of the catheter body; a guidewire lumen 24 extending along the catheter body and the balloon; and a shock wave emitter, generally indicated at 26, inside the balloon (and outside the guidewire lumen). A coupler 30 is connected to a proximal end portion of the catheter body 20. The coupler 30 is configured to couple the control handle 14 to the catheter 16. In the illustrated embodiment, the coupler 30 is configured to electrically and mechanically couple the control handle 14 to the catheter 16. The illustrated coupler 30 includes a first port 32 configured to couple a source of fluid 34 (e.g., saline in a syringe) to the catheter 16 for selectively expanding the balloon, and a second port 36 in communication with the guidewire lumen 24 and configured to receive a guidewire 38 in the guidewire lumen. It is understood that the coupler 30 may be of other configurations and/or designs or the coupler may be omitted and replaced with other components for accomplishing one or more of the functions of the coupler.

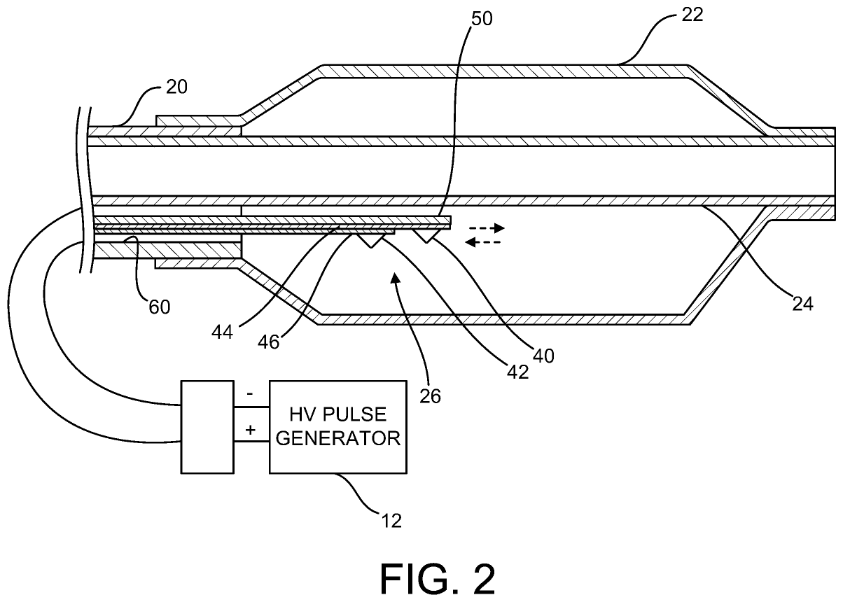

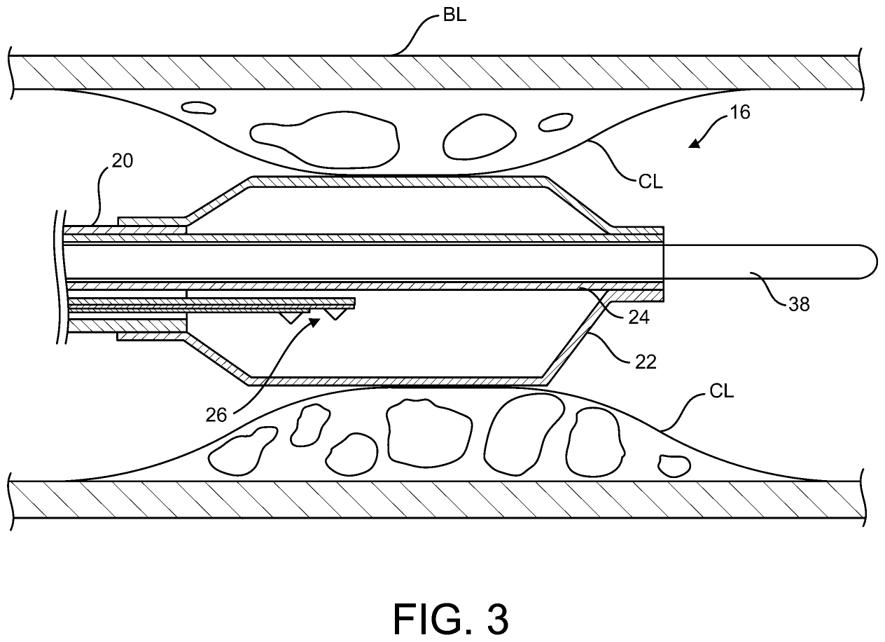

Referring to FIG. 2, the illustrated shock wave emitter 26 includes first and second electrodes 40, 42 electrically connected to the HV generator 12 via first and second electrical conductors 44, 46 (e.g., wires) extending along the catheter body 20 outside the guidewire lumen 24. The electrical conductors 44, 46 are insulated (e.g., insulated sleeves or coatings) and the electrodes 40, 42 are exposed and uninsulated. In the illustrated embodiment, the first electrode 40 is electrically connected to negative terminal of the HV generator 12, and the second electrode 42 is electrically connected to a positive terminal of the generator. The first and second electrodes 40, 42 are spaced apart longitudinally from one another. The longitudinal distance between the electrodes 40, 42 may be fixed. Voltage pulses, for example, from the generator 12 applied to the electrodes 40, 42 produce electrical arcs across the electrodes within the balloon 22. The electrical arcs in the fluid generate shock waves in the fluid. The shock waves propagate through the fluid and the balloon 22 to the calcified lesion where the energy will modify the calcified lesion without the application of excessive pressure by the balloon on the walls of the artery. In one or more embodiments, the first and second electrodes 40, 42 may be of other configurations suitable for producing electrical arcs when the voltage from the generator 12 is applied thereto. In one or more embodiments, more than one pair of first and second electrodes 40, 42 may be provided and spaced apart longitudinally from one another. In such an embodiment, the electrode pairs may be wired in parallel for simultaneous electrical arcs and shock wave generation. In yet another embodiment, explained below in relation to FIG. 3, a shock wave emitter 126 may be unipolar, including a single electrode electrically connected to a HV generator 112.

In one or more embodiments, the shock wave emitter is configured to deliver other types of energy to the fluid in the balloon. For example, the shock wave emitter may include a distal end of an optical fiber that is in communication with a laser or other light source. The shock wave emitter may be other types of shock wave emitters configured to deliver energy to fluid in the balloon suitable to produce a shock wave in the balloon.

Referring still to FIG. 2, the shock wave emitter 26 is selectively movable longitudinally within the balloon 22 to adjust the longitudinal position of the emitter in the balloon. In this way, the location of the generated shock wave (that is, the location of the origin of the generated shock wave) is selectively adjustable so that the practitioner can target specific locations of the calcified lesion, for example. The first and second electrodes 40, 42 are fixedly secured to an actuator 50 that is longitudinally movable to impart longitudinal movement of the shock wave emitter. In the illustrated embodiment, the actuator 50 comprises a control shaft extending along the catheter body 20 outside the guidewire lumen 24. The illustrated control shaft 50 extends to the control handle 14 and is connected to a slider 54 (FIG. 1) on the handle that is operable by the practitioner to impart longitudinal movement of the shock wave emitter 26. The control shaft 50 may include a coiled shaft to provide suitable flexibility and pushability. Referring to FIG. 1, the handle 14 may include indicia, generally indicated at 56, adjacent the slider 54 to indicate the longitudinal position of the shock wave emitter relative to the balloon 22. The indicia 56 may be presented on a graphical user interface in one or more embodiments.

In general, other than the addition of the shock wave emitter 26, the balloon catheter 16 may be suitably constructed similar to a conventional angioplasty balloon catheter. The catheter body 20 and the guidewire lumen 24 may be formed from suitable polymer materials and have suitable flexibility and pushability for navigating vasculature. The balloon 22 may be formed from a suitable polymer material and have suitable compliance and burst pressure, among other parameters.

An inflation lumen 60 is defined between the interior surface of the catheter body 20 and the exterior surface of the guidewire lumen 24. The inflation lumen 60 is in fluid communication with the balloon 22 to deliver the fluid (e.g., saline) from/to the fluid source 34 to/from the balloon for inflating/deflating the balloon. The first and second electrical conductors 44, 46 and the actuator 50 extend along the inflation lumen and are translatable therein. In one or more other embodiments, the first and second electrical conductors 44, 46 and the actuator 50 may be disposed outside the inflation lumen 60.

Referring to FIG. 3, in an exemplary method of use, the balloon catheter 16 is feed along the guidewire 38 through a blood vessel BL, for example, to a calcified lesion CL. The balloon 22 is then expanded so that the balloon contacts the lesion CL. The longitudinal position of the shock wave emitter 26 (e.g., the electrodes 40, 42) relative to the inflated balloon can be adjusted. For example, the practitioner, using fluoroscopy, may adjust the position of the emitter 26 to be adjacent a mass of calcified plaque in the lesion. In another embodiment, the shock wave emitter 26 may be positioned at a proximal location initially and then moved distally along the balloon 22 during treatment to different positions to effect treatment along the lesion. After suitable treatment, the balloon 22 is deflated and the catheter 16 is withdrawn from the blood vessel BL.

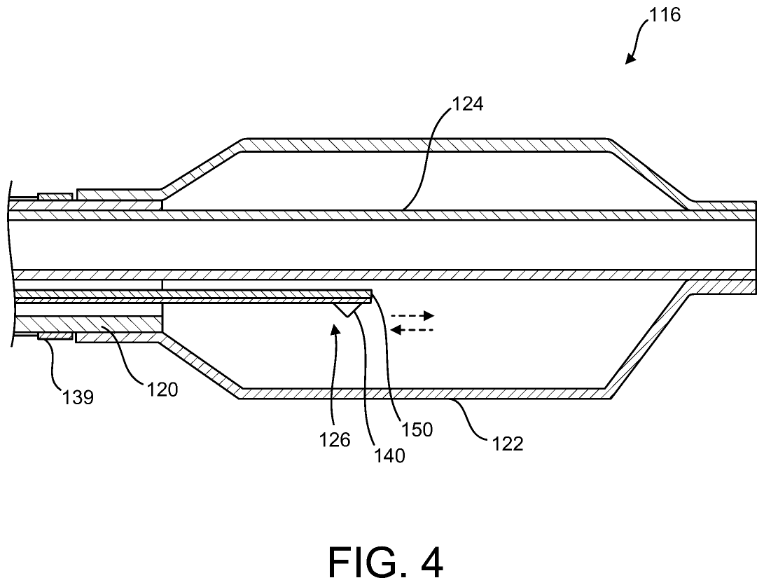

Referring to FIG. 4, another embodiment of an IVL balloon catheter is generally indicated at reference numeral 116. The balloon catheter 116 generally includes an elongate catheter body 120, an expandable balloon 122 (shown expanded in FIG. 4) at the distal end portion of the catheter body, a guidewire lumen 124 extending along the catheter body and the balloon, and a shock wave emitter, generally indicated at 126, inside the balloon (and outside the guidewire lumen). The other components of the system 10 may be incorporated in this embodiment. The difference between the present catheter 116 and the first catheter 16 is the shock wave emitter 126. The shock wave emitter 126 is unipolar or monopolar including a single, unipolar electrode 140. A grounding conductor 139 is coupled to the catheter body 120 at the proximal end portion thereof. The grounding conductor 139 may be electrically connected to ground, such as through a grounding pad. Like the first embodiment, the shock wave emitter 126 is coupled to an actuator 150 to enable a practitioner to adjust the longitudinal position of the emitter within the balloon 122. The teachings of the adjustability of the shock wave emitter related to the first embodiment apply equally to the present embodiment. In one or more embodiments, the shock wave emitter 126 may not be selectively adjustable.

In one or more embodiments, a ceramic insulator may be disposed on the unipolar electrode 140 to enable energy to be focused at a tip of the electrode. In one example, Polyaryletherketone ceramic (e.g., PEEK) may be used. It is believed the unipolar electrode 140 produces a shock wave by generating a plasma arc across an electrolysis bubble. The arc does not extend to the grounding pad (in contrast to a bipolar shock wave emitter 26 of the first embodiment, where the arc extends from the first electrode to the second electrode). When a voltage is applied to the unipolar electrode, a low level of current may flow between the electrode and grounding pad, which may cause dissociation of hydrogen and oxygen in the surrounding fluid such that a gas bubble (e.g., an electrolysis bubble) forms at the exposed, focused tip of the electrode 140. When the applied voltage is increased to a high value (e.g., from about 500 V to about 3000 V), a plasma arc forms at the electrode tip and arcs across the gas bubble to the surrounding fluid. This plasma arc may generate sufficient heat to form a steam bubble in the fluid, the formation of which gives rise to a first shock wave. When the steam bubble collapses, a second shock wave may be formed.

An exemplary use of the IVL catheter balloon 116 is the same as the first embodiment 16, and the teachings set forth above apply equally to the second embodiment.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

The invention may be further described by reference to the following numbered paragraphs:
1. An intravascular lithotripsy balloon catheter system comprising:
   a catheter body configured to be received in a blood vessel of a subject, the catheter body having opposite proximal and distal end portions and a longitudinal axis extending between the proximal and distal end portions;
   an energy source coupled to the catheter body;
   a balloon coupled to the distal end portion of the catheter body, wherein the balloon is configured to receive fluid to expand the balloon; and
   a shock wave emitter in the balloon, the shock wave emitter configured to receive energy from the source of energy and configured to use the received energy to create a shock wave that propagates through the fluid in the balloon,
   wherein the shock wave emitter is selectively movable longitudinally within the balloon to adjust a longitudinal position of the shock wave emitter relative to the balloon.
2. The intravascular lithotripsy balloon catheter system set forth in paragraph 1, further comprising an actuator operatively coupled to the shock wave emitter, wherein the actuator is configured to impart longitudinal movement of the shock wave emitter relative to the balloon.
3. The intravascular lithotripsy balloon catheter system set forth in paragraph 2, wherein the actuator includes an actuator shaft extending along the catheter body.
4. The intravascular lithotripsy balloon catheter system set forth in paragraph 3, further comprising a control handle coupled to the proximal end portion of the catheter body, wherein the control handle includes a slider operatively connected to the actuator and configured to selectively operate the actuator to impart longitudinal movement to the shock wave emitter.
5. The intravascular lithotripsy balloon catheter system set forth in paragraph 1, wherein the energy source includes an electrical energy source, wherein the shock wave emitter comprises at least one electrode, wherein the at least one electrode is configured to produce an electrical arc when electrical energy from the electrical energy source is applied to the at least one electrode thereby creating a shock wave within the balloon.
6. The intravascular lithotripsy balloon catheter system set forth in paragraph 5, further comprising an inflation lumen extending along and within the catheter body, wherein the inflation lumen is in fluid communication with the balloon and configured to deliver fluid to the balloon the expand the balloon.
7. The intravascular lithotripsy balloon catheter system set forth in paragraph 6, wherein the actuator is disposed in the inflation lumen.
8. The intravascular lithotripsy balloon catheter system set forth in paragraph 6, further comprising a guidewire lumen disposed in the catheter body and the balloon, the guidewire lumen configured to receive a guidewire therein.
9. The intravascular lithotripsy balloon catheter system set forth in paragraph 8, wherein the shock wave emitter is disposed outside the guidewire lumen.
10. The intravascular lithotripsy balloon catheter system set forth in paragraph 5, wherein the at least one electrode comprises first and second electrodes configured to produce the electrical arc therebetween.
11. The intravascular lithotripsy balloon catheter system set forth in paragraph 10, wherein the first and second electrodes are configured to move together longitudinally.
12. The intravascular lithotripsy balloon catheter system set forth in paragraph 5, wherein the at least one electrode includes a unipolar electrode.
13. The intravascular lithotripsy balloon catheter system set forth in paragraph 12, further comprising a grounding conductor coupled to the distal end portion of the catheter body and configured to be connected to ground.
14. The intravascular lithotripsy balloon catheter system set forth in paragraph 13, further comprising ceramic insulation disposed on the unipolar electrode.
15. An intravascular lithotripsy balloon catheter comprising:
   a catheter body configured to be received in a blood vessel of a subject, the catheter body having opposite proximal and distal end portions and a longitudinal axis extending between the proximal and distal end portions;
   an electrical energy source coupled to the catheter body;
   a balloon coupled to the distal end portion of the catheter body, wherein the balloon is configured to receive fluid to expand the balloon; and
   a shock wave emitter in the balloon, the shock wave emitter including a unipolar electrode in communication with the electrical source of energy and configured to deliver energy from the electrical energy source to the fluid in the balloon thereby creating a shock wave within the balloon,
   wherein a ceramic insulation is disposed on the unipolar electrode to focus energy at a tip of the unipolar electrode.
16. The intravascular lithotripsy balloon catheter set forth in paragraph 15, wherein the ceramic insulation comprises a Polyaryletherketone ceramic material.
17. The intravascular lithotripsy balloon catheter set forth in paragraph 16, further comprising a grounding conductor for the shock wave emitter coupled to the proximal end portion of the catheter body and configured to be connected to ground.
18. An intravascular lithotripsy balloon catheter comprising:
   a catheter body configured to be received in a blood vessel of a subject, the catheter body having opposite proximal and distal end portions and a longitudinal axis extending between the proximal and distal end portions;
   a balloon coupled to the distal end portion of the catheter body, wherein the balloon is configured to receive fluid to expand the balloon;
   a shock wave emitter received in the balloon, the shock wave emitter comprising a unipolar electrode, wherein the unipolar electrode is configured to produce an electrical arc when a voltage is applied to the at least one electrode thereby creating a shock wave within the balloon; and
   a grounding conductor for the shock wave emitter coupled to the proximal end portion of the catheter body and configured to be connected to ground.
19. The intravascular lithotripsy balloon catheter set forth in paragraph 18, wherein the grounding conductor is configured to be connected to a grounding pad.
20. The intravascular lithotripsy balloon catheter set forth in paragraph 19, wherein a ceramic insulation is disposed on the unipolar electrode to focus energy at a tip of the unipolar electrode.

1-15. (canceled)

16. An intravascular lithotripsy balloon catheter system comprising:
a catheter body configured to be received in a blood vessel of a subject, the catheter body having opposite proximal and distal end portions and a longitudinal axis extending between the proximal and distal end portions;
an energy source coupled to the catheter body;
a balloon coupled to the distal end portion of the catheter body, wherein the balloon is configured to receive fluid to expand the balloon; and
a shock wave emitter in the balloon, the shock wave emitter configured to receive energy from the source of energy and configured to use the received energy to create a shock wave that propagates through the fluid in the balloon,
wherein the shock wave emitter is selectively movable longitudinally within the balloon to adjust a longitudinal position of the shock wave emitter relative to the balloon.

17. The intravascular lithotripsy balloon catheter system set forth in claim 16, further comprising an actuator operatively coupled to the shock wave emitter, wherein the actuator is configured to impart longitudinal movement of the shock wave emitter relative to the balloon.

18. The intravascular lithotripsy balloon catheter system set forth in claim 17, wherein the actuator includes an actuator shaft extending along the catheter body.

19. The intravascular lithotripsy balloon catheter system set forth in claim 18, further comprising a control handle coupled to the proximal end portion of the catheter body, wherein the control handle includes a slider operatively connected to the actuator and configured to selectively operate the actuator to impart longitudinal movement to the shock wave emitter.

20. The intravascular lithotripsy balloon catheter system set forth in claim 16, wherein the energy source includes an electrical energy source, wherein the shock wave emitter comprises at least one electrode, wherein the at least one electrode is configured to produce an electrical arc when electrical energy from the electrical energy source is applied to the at least one electrode thereby creating a shock wave within the balloon.

21. The intravascular lithotripsy balloon catheter system set forth in claim 20, further comprising an inflation lumen extending along and within the catheter body, wherein the inflation lumen is in fluid communication with the balloon and configured to deliver fluid to the balloon the expand the balloon.

22. The intravascular lithotripsy balloon catheter system set forth in claim 21, wherein the actuator is disposed in the inflation lumen.

23. The intravascular lithotripsy balloon catheter system set forth in claim 21, further comprising a guidewire lumen disposed in the catheter body and the balloon, the guidewire lumen configured to receive a guidewire therein.

24. The intravascular lithotripsy balloon catheter system set forth in claim 23, wherein the shock wave emitter is disposed outside the guidewire lumen.

25. The intravascular lithotripsy balloon catheter system set forth in claim 20, wherein the at least one electrode comprises first and second electrodes configured to produce the electrical arc therebetween.

26. The intravascular lithotripsy balloon catheter system set forth in claim 25, wherein the first and second electrodes are configured to move together longitudinally.

27. The intravascular lithotripsy balloon catheter system set forth in claim 20, wherein the at least one electrode includes a unipolar electrode.

28. The intravascular lithotripsy balloon catheter system set forth in claim 27, further comprising a grounding conductor coupled to the distal end portion of the catheter body and configured to be connected to ground.

29. The intravascular lithotripsy balloon catheter system set forth in claim 28, further comprising ceramic insulation disposed on the unipolar electrode.

30. An intravascular lithotripsy balloon catheter comprising:
a catheter body configured to be received in a blood vessel of a subject, the catheter body having opposite proximal and distal end portions and a longitudinal axis extending between the proximal and distal end portions;
an electrical energy source coupled to the catheter body;
a balloon coupled to the distal end portion of the catheter body, wherein the balloon is configured to receive fluid to expand the balloon; and
a shock wave emitter in the balloon, the shock wave emitter including a unipolar electrode in communication with the electrical source of energy and configured to deliver energy from the electrical energy source to the fluid in the balloon thereby creating a shock wave within the balloon,
wherein a ceramic insulation is disposed on the unipolar electrode to focus energy at a tip of the unipolar electrode.

31. The intravascular lithotripsy balloon catheter set forth in claim 30, wherein the ceramic insulation comprises a Polyaryletherketone ceramic material.

32. The intravascular lithotripsy balloon catheter set forth in claim 31, further comprising a grounding conductor for the shock wave emitter coupled to the proximal end portion of the catheter body and configured to be connected to ground.

33. An intravascular lithotripsy balloon catheter comprising:
a catheter body configured to be received in a blood vessel of a subject, the catheter body having opposite proximal and distal end portions and a longitudinal axis extending between the proximal and distal end portions;
a balloon coupled to the distal end portion of the catheter body, wherein the balloon is configured to receive fluid to expand the balloon;
a shock wave emitter received in the balloon, the shock wave emitter comprising a unipolar electrode, wherein the unipolar electrode is configured to produce an electrical arc when a voltage is applied to the at least one electrode thereby creating a shock wave within the balloon; and
a grounding conductor for the shock wave emitter coupled to the proximal end portion of the catheter body and configured to be connected to ground.

34. The intravascular lithotripsy balloon catheter set forth in claim 33, wherein the grounding conductor is configured to be connected to a grounding pad.

35. The intravascular lithotripsy balloon catheter set forth in claim 34, wherein a ceramic insulation is disposed on the unipolar electrode to focus energy at a tip of the unipolar electrode.
